(19) United States
(12) Patent Application Publication
(10) Pub. No.: US 2009/0246074 A1
(43) Pub. Date: Oct. 1, 2009

(54) System and method for sterilizing a processing line

(75) Inventors: Philip E. Nelson, Pellston, MI (US); Yingchang Han, Dublin, OH (US)

Correspondence Address: Mr. Edward J. Timmer, P.O. Box 770, Richland, MI, 49083-0770, US

(73) Assignee: Purdue Research Foundation

(21) Appl. No.: 12/316,912

(22) Filed: Dec. 17, 2008

Related U.S. Application Data
(60) Provisional application No. 61/008,411, filed on Dec. 20, 2007.

(51) Int. Cl. A61L 9/015; A61L 9/03

(52) U.S. Cl. 422/29; 422/292

(57) ABSTRACT

The invention comprises a system and method for sterilizing an aseptic processing line comprising the steps of generating chlorine dioxide gas, introducing the chlorine dioxide gas inside the aseptic processing line as a sterilization step, and removing the chlorine dioxide gas from the aseptic processing line. The aseptic processing line comprises at least one processing apparatus such as a filter, heater, cooler, filler, surge tank, and/or packager connected to a piping network.

This application claims priority and benefits of provisional application Ser. No. 61/008,411 filed Dec. 20, 2007, the disclosure of which is incorporated herein by reference.

### BACKGROUND OF THE INVENTION

1. Field of the Invention

The present invention relates to a system and method for sterilizing a processing line and, in particular, using gaseous chlorine dioxide to sterilize an aseptic processing line.

2. Description of Related Art

Chlorine dioxide (ClO₂) is a strong oxidizing and antimicrobial agent. It has been reported to effectively inactivate bacteria, including pathogens, viruses, bacterial spores, and algae. It has about 2.5 times the oxidation capacity of chlorine (Bernarde at al. 1965). Advantages of ClO₂ over chlorine also include lack of odor and taste, effectiveness at low concentration, non-conversion to chlorophenols which result in residual smells and flavors, ability to remove chlorophenols already present from other sources, and inability to form harmful chloramines and THMs (Elphick 1998).

Gaseous ClO₂ has been shown to be an effective disinfectant especially related to use in the medical sciences. Rosenblatt et al. (U.S. Pat. No. 4,504,442, U.S. Pat. No. 4,681,739) reported use of gaseous ClO₂ to sterilize the gas-impermeable surfaces of implements used in the medical sciences, such as those made of porcelain, ceramics, metal, plastics, and glass. In these patents, treatment with 10 to 40 mg/L ClO₂ gas at room temperature and high relative humidity demonstrated sporicidal action. Jeng and Woodworth (1990) also reported the sporicidal activity of ClO₂ gas under square-wave conditions within an experimental sterilizer used for medical implements. Heredia et al. (U.S. Pat. No. 6,235,240) disclosed an apparatus and methods for generating, administering, extracting and recovering sterilant gas (ClO₂ gas) for sterilization and/or decontaminating microbial isolators, The sterilization of aseptic fill isolators and vessels using chlorine dioxide gas in pharmaceutical industry has also been evaluated (Eylath et al. 2003).

ClO₂ can be used to sanitize food contact surfaces and food surfaces as a gas or in an aqueous form in the food industry. Aqueous ClO₂ has been approved for use in washing fruits and vegetables in an amount not to exceed 3 ppm residual ClO₂, treatment of poultry processing water, and sanitation of processing equipment (FDA). Han et al. (1999) have demonstrated that gaseous ClO₂ is highly effective for sanitation or sterilization of aseptic juice storage tank. This study has shown that up to 7 log cfu spoilage microorganisms inoculated to the surface of a model storage tank are completely inactivated under a treatment by 10 mg/L ClO₂ gas for 30 min. at 9-28° C. and above 90% relative humidity (RH). The effectiveness of ClO2 gas against microorganisms may increase with the increase of ClO₂ gas concentration, exposure time, relative humidity, and temperature (Han et al. 2001). ClO₂ gas has also been shown to be effective (more than 5-log reduction) at reducing microorganisms on produce surfaces, such as Salmonella spp. on strawberries (Han et al. 2004), Listeria monocytogenes and E. coli O157:H7 on green pepper surfaces (Han et al. 2000a and b, 2001 a and b) and apples (Du et al. 2002, 2003), E. coli on apples (Sapers et al. 2003), and Listeria monocytogenes, E. coli O157:H7 and Salmonella Typhimurium on lettuce leaves (Lee et al. 2004).

Aseptic processing and packaging is a continuous operation that is used to produce a commercially sterile product contained in a hermetically sealed container. This technology has been widely used in food and pharmaceutical industries. A typical aseptic processing and packaging system is shown in FIG. 1. The aseptic processing system can conveniently include several elements: the raw product inlet; flow control; product heating; hold tube; product cooling; and packaging (Chambers and Nelson, 1992). To establish an aseptic process system, processing equipment and downstream piping, products, packaging materials, and packaging equipment must be sterilized and the sterile condition must be maintained throughout the whole system. It is critical that the process system and product filler be thoroughly sterilized before the sterilization and packaging of products.

Currently hot water or saturated steam is used to sterilize the processing line and packaging facilities before production. It usually takes about two hours to heat up the whole line. The sterilization using steam and hot water not only costs a great amount of energy, but also provides inadequate sterility on some places, such as dead corners or hard-to-reach surfaces on pipes, gaskets, valves, pumps, and vessels, where biofilms commonly grow and attach to. A biofilm is a layer of microorganisms contained in a matrix (slime layer), which forms on surfaces in contact with water. Incorporation of pathogens in biofilms can protect the pathogens from concentrations of biocides that would otherwise kill or inhibit those organisms freely suspended in water. Biofilms provide a safe haven for organisms like Listeria, E. coli and legionella where they can reproduce to levels where contamination of products passing through that water becomes inevitable.

The use of chlorine dioxide gas to sterilize aseptic juice storage tank surfaces has been studied (Han et at. 1999). The sterilization of aseptic fill isolators and vessels using chlorine dioxide gas in pharmaceutical industry has also been evaluated (Eylath et al. 2003). However, using gaseous chlorine dioxide to sterilize an aseptic processing line has not been previously studied.

An object of the present invention is to provide a system and method for sterilizing a processing line using gaseous chlorine dioxide that overcomes the disadvantages of conventional sterilization procedures.

### SUMMARY OF THE INVENTION

This invention sterilizes an aseptic processing line using an effective amount of chlorine dioxide gas to this end.

An illustrative embodiment of the processing system and method involves sterilizing an aseptic processing line comprising the steps of generating chlorine dioxide gas, introducing the chlorine dioxide gas inside the aseptic processing line as a sterilization step, and removing the chlorine dioxide gas from the aseptic processing line. The aseptic processing line can comprise at least one processing apparatus such as a filter, heater, cooler, product filler, surge tank, and/or packager connected by a piping network. Bleeders can be used to flow gas into corners and dead spaces in the processing line.

The generating of chlorine dioxide gas can be from a generator or a chlorine dioxide solution vaporized with a carrier gas such as air or nitrogen.

An embodiment of the invention comprises connecting the aseptic line in a closed chlorine dioxide gas treatment loop prior to the sterilization step. The chlorine dioxide gas can reduce the microorganisms in the closed loop environment.

The sterilization step can have a time range of about 30 minutes to about 1 hour. The atmosphere in the aseptic processing line during the sterilization step can have a concentration of chlorine dioxide between about 1 to about 30 mg/L, a relative humidity between about 80 to about 95%, and a temperature between about 4 to about 45° C. The relative humidity in the line can be adjusted. The chlorine dioxide gas can also be circulated in the line during the sterilization step. In a preferred embodiment, the atmosphere in the aseptic processing line can have a concentration of chlorine dioxide between about 3 to about 10 mg/L, a relative humidity above 85%, and a temperature between about 3 to about 25° C.

The chlorine dioxide gas is removed after the sterilization step. The removal step can comprise flowing a secondary gas such as nitrogen, air, or a mixture thereof into the line, using a blower to blow the chlorine dioxide gas from the line, and/or flowing water such as heated water through the line.

The chlorine dioxide concentration in the line can be monitored during the various steps.

After sterilization of the line, the chlorine dioxide gas can be recycled using a recycling device or neutralized using a neutralizing device. The chlorine dioxide can flow through a reducing agent such as soda lime, sodium thiosulfate, sodium sulfite, or a mixture thereof, or through gas scrubber. Flowing of sterile air through the line, after the flowing and removal steps, can maintain the sterilized surfaces therein.

### DESCRIPTION OF DRAWINGS

FIG. 1 is a schematic view of a conventional aseptic processing and packaging system for processing a raw product.

FIG. 2 is a schematic view of a gaseous sterilization system for an aseptic processing line with a surge tank pursuant to the invention.

FIG. 3 is a schematic view of a gaseous sterilization system for an aseptic processing line without a surge tank pursuant to the invention.

FIG. 4 is a side view of a chlorine dioxide gas generation and scrub system using a conventional chlorine dioxide gas generator.

FIG. 5 is a side view of a chlorine dioxide gas generation and scrub system using a solution to generate the chlorine dioxide gas.

### DETAILED DESCRIPTION OF THE INVENTION

The invention is especially useful for sterilization of aseptic processing systems for food, pharmaceutical, biological, chemical products, or other raw products, but is not limited thereto, as sterilization of other processing systems can benefit from this invention. The aseptic processing line can be made of a series of filters, heaters, coolers, packaging, and other processing equipment necessary for the finished product connected via a piping network.

The invention provides a system and method for sterilizing aseptic processing and packaging lines for raw products using ClO₂ gas as a sterilant. In an illustrative embodiment offered for purposes of illustration and not limitation, the method and system can involve generating ClO₂ gas, circulating the ClO₂ gas inside of the aseptic processing line, removing ClO₂ gas with filtrated air or water, and recycling or neutralizing the ClO₂ solution. The sterilization process can be applied after CIP (Cleaning-in-place) cleaning and provide sterile or aseptic environment inside the processing and packaging lines.

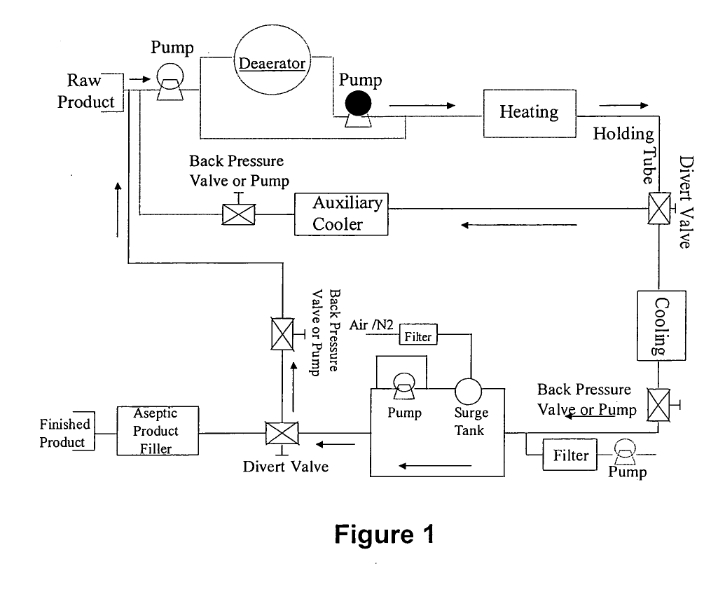

Compared to a conventional steam sterilization system, as shown in FIG. 1, the ClO₂ gas sterilization of an aseptic processing system can have several advantages. The invention can provide a higher efficacy in inactivation of microorganisms, which is especially for biofilms and bacteria spores. There is much less energy cost and may annually save one million dollars for an aseptic processing line. There is less sterilization time (one half-hour to one hour reduction). The invention can also advantageously sterilize air filters in the line. Currently, the air filters need to be sterilized separately using steam.

A further illustrative embodiment of the invention involves a method and system for sterilizing an aseptic processing line comprising the steps of generating chlorine dioxide gas, flowing the chlorine dioxide gas inside the aseptic processing line as a sterilization step, and removing the chlorine dioxide gas from the aseptic processing line.

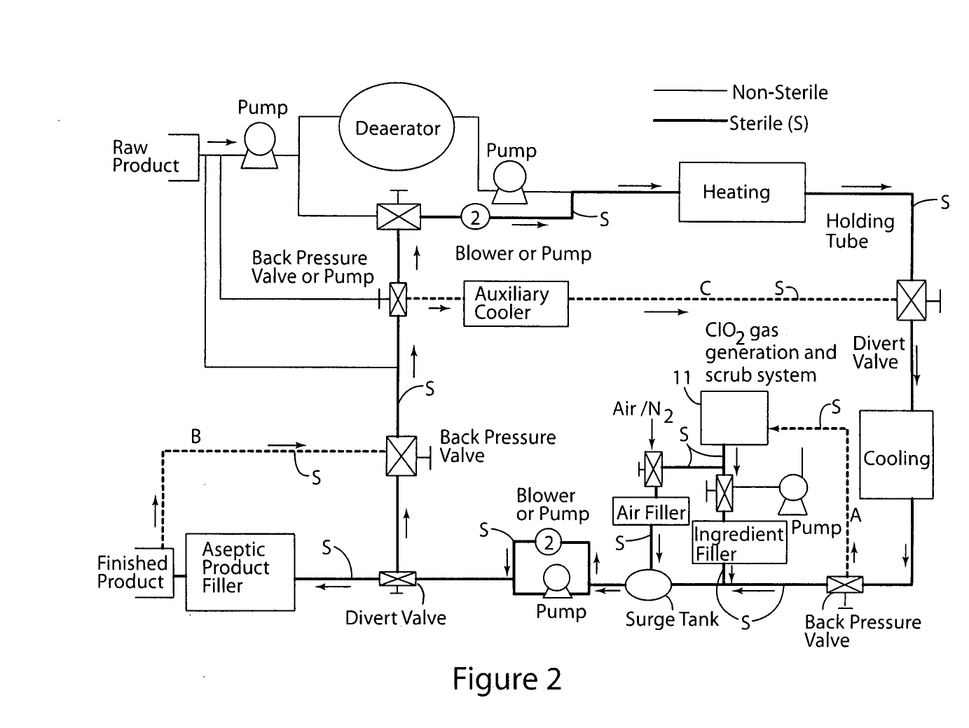

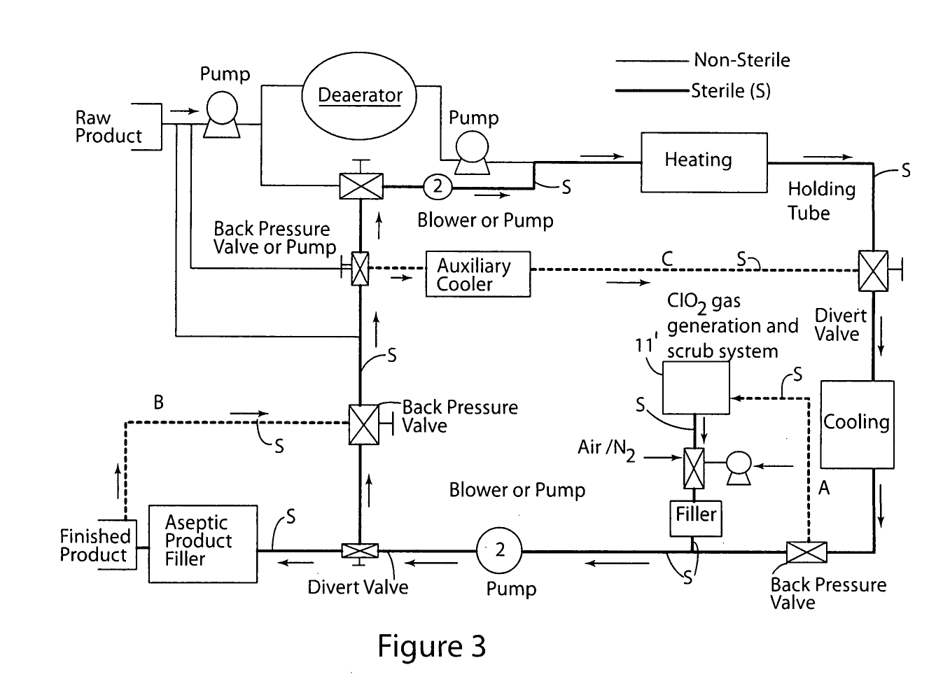

The aseptic processing line comprises at least one processing apparatus such as a filter, heater, cooler, aseptic product filler, surge tank, and/or packager connected to a piping network for receiving and processing a raw product in a manner to provide a sterile product, FIGS. 2 and 3. The filters can be air or other type of gas filters or a water or product type filter. The heater, cooler, and product filler can be a conventional heater, cooler, and product filler used in a conventional aseptic processing line. The surge tank is a conventional storage tank that is typically used to temporarily store sterile product that can be used to provide a continuous supply of product to the filler or to divert sterile product in the event of line stoppage. The packager can be a conventional packaging equipment that provides a sterile package and environment suitable for the processed product. The piping network can be conventional piping used in an aseptic processing line. In addition, the line can have bleeders to flow chlorine dioxide gas into the comers and dead spaces of the processing line. The aseptic processing line can include multiple processing apparatus depending on the processing requirements for the processed product. The entire aseptic processing line can be sterilized or each processing apparatus can be sterilized separately depending on the sterilization needs.

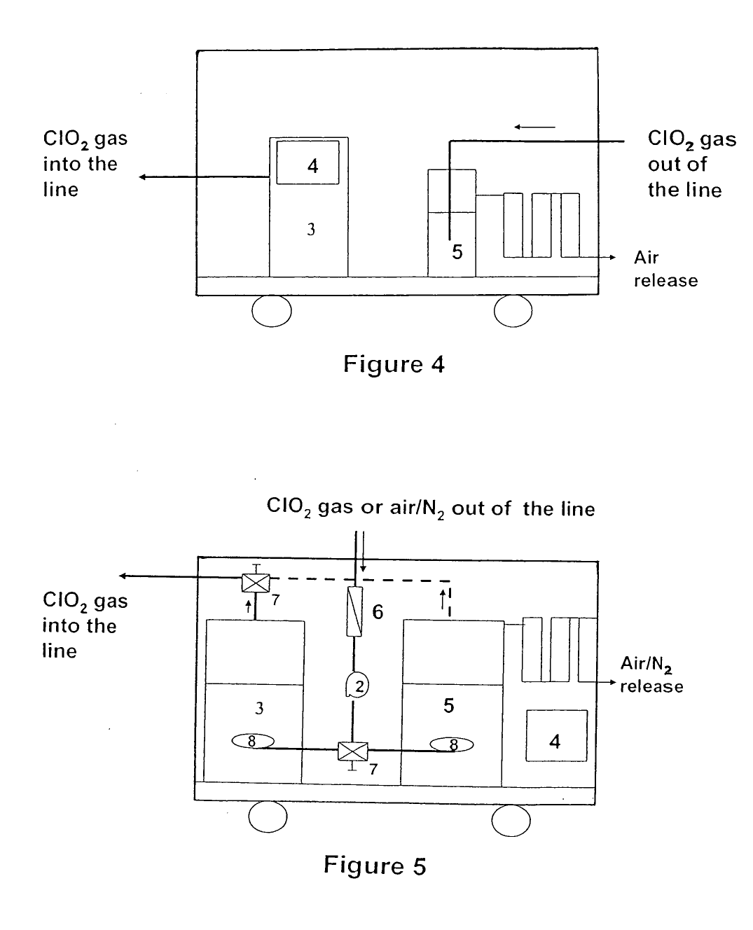

The generating of chlorine dioxide gas can be from a gas generator or a device where chlorine dioxide solution is vaporized with a carrier gas such as air or nitrogen. Two ClO₂ gas generation and scrub systems are shown in FIGS. 4 and 5. Each system is designed and installed as one unit that has the ability to generate, monitor, and recycle or neutralize ClO₂ gas. As shown in FIG. 4, ClO₂ gas can be generated with any type of commercial ClO₂ gas generators, such as a CDG ClO₂ gas generator (CDG Technology, Inc., Bethlehem, Pa.) or a ClorDiSys ClO₂ gas generator (ClorDiSys Solutions, Inc. Lebanon, N.J.). FIG. 4 shows the chlorine dioxide generator 3, the gas monitor 4, and the scrubber 5. The systems shown in FIGS. 4 and 5 can also continuously monitor ClO₂ gas concentration. FIG. 5 shows the device where ClO₂ or vapor gas is generated when a ClO₂ solution is vaporized with nitrogen or air. A ClO₂ solution can be used with 1-10 g/L ClO₂ that can be prepared by any of the reported methods or commercially purchased in its stabilized phase, such as acidified solution or frozen state. FIG. 5 shows the chlorine dioxide vaporizer 3, monitor 4, vaporizer and scrubber 5, air filter 6, divert valve 7, and air disperser 8. With a pump or blower 2, air or nitrogen in the line is introduced into the vaporizer 3 through the air disperser 8. ClO₂ can be stripped off and circulated in the aseptic line. The temperature of ClO₂ solution is important because it affects the vaporization rate of ClO₂. The solubility of ClO₂ increases as the temperature decreases. The solubility of the ClO₂ can be between about 3-8 g/L depending on the temperature and pressure. The solubility of ClO₂ increases as the temperature decreases. For example, the solubility of ClO₂ can be up to 8 g/L at 20° C. and 2.63 g/L at 40° C. under 74.9 mbar partial pressure.

FIG. 2 shows sterilization of an aseptic system using a closed ClO₂ gas treatment loop using chlorine dioxide gas. Any water in the process line should be totally drained because high water residue may reduce the effectiveness of the treatment due to the highly water-soluble nature of ClO₂. For sterilization, ClO₂ gas is generated from a gas generation and scrub system 11. The gas is introduced to the aseptic process line through two filters: air/nitrogen filter and ingredient filter. These filters are conventionally pursuant to past practice sterilized with steam separately. In this invention, the filters advantageously can be sterilized in line. After flowing through the filters, ClO₂ gas is introduced into the top of a surge tank. For a large size of surge tank, a fan is needed to ensure adequate distribution of the gas inside of the tank. The gas is then taken out from the bottom of the tank using a blower or pump 2. A bypass may be needed for the product pump if the pump provides too much resistance to the gas flow. The gas continuously flows through several divert valves, is further driven by a second air blower or pump 2, and then enters into a heating device, as indicated by the bold line and arrows in FIG. 2. From the heating device, the gas continuously flows through a holding tube, a divert valve, a cooling unit, and a back pressure valve, and then goes back to the surge tank to make a circulation. After circulation for preselected amount of time, preferably about 30 minute to about 1 hour, the gas is introduced back to the gas generation and scrub system 1 through the backpressure valve, as indicated by the dotted line A in FIG. 2. At the same time, introduction of ClO₂ gas stops and air/nitrogen gas is fed into the system through the air filter to flush the residual ClO₂ out of the system and to maintain a positive pressure and sterility in the system.

Sterilization of an aseptic processing system without a surge tank (FIG. 3) using a closed ClO₂ gas treatment loop is similar to, but less complex than a system with a surge tank (FIG. 2). The only difference is at the introduction position of ClO₂ gas from the generation system 11′. ClO₂ gas is introduced through the ingredient filter. After the sterilization, air or nitrogen is also introduced through this filter. Because there is no surge tank in this system, the amount of ClO₂ gas used is less than that for the system with a surge tank. The sterilization time may also be shorter. The gas concentration should be monitored at the entrance of the ingredient filter and after cooling.

The above embodiments of the invention involve connecting the aseptic processing line in a closed ClO₂ gas treatment loop prior to the cold-sterilization step. The closed loop environment may include a venting device for exhausting ClO₂ gas. The chlorine dioxide gas can reduce the microorganisms in the closed loop environment. In practice of one embodiment of the invention, the method can be used to sterilize the aseptic product filler wherein the ClO₂ gas flows through the divert valve shown and is introduced into the aseptic product filler that is placed in or connected to the closed loop environment with a venting device. The leaked ClO₂ gas from the product filler can also provide a benefit to clean up the environment around the product filler. The ClO₂ gas in the product filler flows back to the processing line through a backpressure valve, as indicated by a dotted line B in FIG. 2.

Also shown in FIG. 2, the auxiliary cooler can also be sterilized by the ClO₂ gas, as indicated by a dotted line C in FIG. 2. In addition, bleeders are needed to ensure sterilization of some dead spots or comers.

Sterilization treatment includes the following factors: the concentration of ClO₂ gas in line, gas exposure time, relative humidity, temperature, types of target (resistant) microorganisms (spoilage microbes and spores), and size of the surge tank. The ClO₂ gas concentration, exposure time, relative humidity, and temperature are the most important four factors. The sterilization step can have a time period of about 30 minutes to about 1 hour. The atmosphere in the aseptic processing line during the sterilization step can a concentration of chlorine dioxide between about 1 to about 30 mg/L, a relative humidity between about 80 to about 95%, and a temperature between about 4 to about 45° C. The relative humidity in the line can be adjusted by flowing steam therein. The chlorine dioxide gas can also be circulated in the line during the sterilization step to circulate the atmosphere to all surfaces in the line. Because the effectiveness of ClO₂ gas against microorganisms may increase with the increase of ClO₂ gas concentration, exposure time, relative humidity, and temperature, a preferred embodiment of the invention can provide an atmosphere in the aseptic processing line during the sterilization step having a concentration of chlorine dioxide between about 3 to about 10 mg/L, a relative humidity above 85%, and a temperature between about 3 to about 25° C.

The chlorine dioxide gas is removed after the sterilization step. The removal step can comprise flowing a secondary gas such as nitrogen, air, or a mixture thereof into the line, using a blower to blow the chlorine dioxide gas from the line, and/or flowing water such as heated water through the line.

The chlorine dioxide concentration in the line can be monitored as needed or continuously during the various steps.

After sterilization of the line the chlorine dioxide gas can be recycled using a recycling device or neutralized using a neutralizing device as shown in FIGS. 4 and 5. The chlorine dioxide can flow through a reducing agent such as soda lime, sodium thiosulfate, sodium sulfite, or a mixture thereof, or through gas scrubber. The ClO₂ gas can be neutralized when it flows through several columns packed with a reducing agent, such as soda lime, sodium thiosulfate, or sodium sulfite, or using a commercial scrubber, such as a CDG ClO₂ gas scrubber (CDG Technology, Inc.). The concentration of ClO₂ gas in the scrubbed air should be less than 0.1 ppm to meet the regulation by the Occupational Safety and Health Administration. The chlorine dioxide can alternately flow into a recycling device such as a dissolving tank, where the chlorine dioxide can be dissolved in cold water at 0-5° C. The recycled chlorine dioxide solution can be further stored in a sanitizer tank and used for other sanitation.

The concentration of gaseous and aqueous ClO₂ may be determined by any of the standard methods, such as DPD-glycine calorimetric method (EPA approved), chlorophenol red calorimetric method, iodometric titration method, amperometric titration method (EPA approved), and direct absorbance method (Greenberg, et al. 1992). The preferred method is the DPD-glycine calorimetric method that can measure both gaseous and aqueous ClO₂ concentration. To measure ClO₂ gas concentration in nitrogen or air, a sample is prepared as follows. Using a 25 ml gas-sampling syringe, 15 ml ClO₂ gas mixture can be sampled and immediately dissolved in 15 ml deionized and neutralized water. Before injecting the gas into the water, some water is repeatedly drawn in and out the syringe to dissolve the gas completely. A DPD colorimetric analysis kit (CHEMetrics, Inc., Calverton, Va., USA) or a spectrophotometer can be used to test ClO₂ concentration. To measure the low amount (<1 ppm) of residual ClO₂ in foods, the amperometric titration method is preferably used. ClO₂ gas concentration may be continuously monitored using the direct absorbance method at 350-450 nm.

After the treatment, filtered air is used to remove residual ClO₂ gas in line. If necessary, a small amount of water is heated in the heater and flow through the whole line to further reduce residues of ClO₂ and its by-products prior to processing of products. In addition, flowing of sterile air through the line, after the flowing and removal steps, can maintain the sterilized surfaces in the line.

It is to be understood that the invention has been described with respect to certain specific embodiments thereof for purposes of illustration and not limitation. The present invention envisions that modifications, changes, and can be made therein without departing from the spirit and scope of the invention as set forth in the following claims.

### REFERENCES

Benarde, M. A., Israel, B. M., Oliveri, V. P., and Granstrom, M, L. 1965. Efficiency of chlorine dioxide as a bactericide. Appl. Microbiol. 13:776.

Chambers, J. V. and Nelson, P. E. 1992. Principles of aseptic processing and packaging. The Food Processors Institute. Washington D.C.

Du, J., Y. Han, and R, H, Linton. 2003. Efficacy of chlorine dioxide gas in reducing Escherichia coli O157:H7 on apple surfaces. Food Microbiology. 20:583-591.

Du J., Y. Han, and R. H. Linton. 2002. Inactivation of Listeria monocytogenes spotted onto different apple surfaces using chlorine dioxide gas. Food Microbiology. 19:481-490. Elphick, A. 1998. The growing use of chlorine dioxide. Processing. March 24.

Eylath, A., Donald L. Wilson, David Thatcher and Anthony Pankau. Successful Sterilization Using Chlorine Dioxide Gas: Part One: Sanitizing an Aseptic Fill Isolator. BioProcess Int'l, July 2003

Eylath, A. Sushil Madhogarhia, Emilio Rivera, Paul Lorcheim and Mark Czarneski. Successful Sterilization Using Chlorine Dioxide Gas: Part Two: Cleaning Process Vessels. BioProcess Int'l, August 2003

FDA, 21 CFR 173.300, 21 CFR 173.325, 21 CFR 178.1010.

Greenberg, A. E., Clesceri, L. S. and Eaton, A. D. (1992) 4500-ClO2 C. Amperometric method I. In Standard methods for the examination of water and wastewater (18^{th} edition) pp. 4-55 and 456. The American Public Health Association, Washington, D.C.

Han, Y., Floros, J. D, Linton, R. H., Nielsen, S. S., and Nelson, P. E. 2001 a. Response surface modeling for the inactivation of Escherichia coli O157: H7 on green peppers (Capsicum annuum L.) by chlorine dioxide gas treatments. 1 Food Protection 64: 1128-1133.

Han, Y., R. H. Linton, S. S. Nielsen, and P. E. Nelson. 2001b. Reduction offisteria monocytogenes on green peppers (Capsicum annuum) by gaseous and aqueous chlorine dioxide and water washing, and its growth at 7° C. J Food Prot. 64:1730-1738.

Han, Y., R. H. Linton, S. S. Nielsen, and P. E. Nelson. 2000a. Inactivation of Escherichia coli O157: H7 on surface-uninjured and -injured green pepper (Capsicum annuum L.) by chlorine dioxide gas as demonstrated by confocal laser scanning microscopy. Food Microbiology. 17(6): 643-655.

Han, Y., D. M. Sherman, R. H. Linton, S. S. Nielsen, and P. E. Nelson. 2000b. The effects of washing and chlorine dioxide gas on survival and attachment of Escherichia coli O157: H7 to green pepper surfaces. Food Microbiology. 17 (5): 521-533.

Han, Y., Guentert, A. M., Smith, R. S., Linton, R. H. and Nelson, P. E. 1999. Efficacy of chlorine dioxide gas as a sanitizer for tanks used for aseptic juice storage. Food Microbiol. 16: 53-61.

Hereedia, L. M., Battisti, D. L., and Plocic, M. F. 2001. Gas recovery system. U.S. Pat. No. 6,235,240.

Jeng, D. K. and Woodworth, A. G. 1990. Chlorine dioxide gas sterilization under square-wave conditions. Appl. Environ. Microbiol. 56: 514-519

Lee, S.; Costello, M; & Kang, D. 2004. Efficacy of chlorine dioxide gas as a sanitizer of lettuce leaves. J. Food Prot. 2004; 67(7): 1371-1376.

Rosenblatt, D. H., Rosenblatt, A. A. and Knapp, J. E. Use of chlorine dioxide gas as a chemosterilizing agent. U.S. Pat. No. 4,504,442 (1985) and U.S. Pat. No. 4,681,739 (1987).

Sapers,-G -M; Walker,-P -N; Sites,-J -E; Annous,-B -A; Eblen,-D -R. 2003. Vapor-phase decontamination of apples inoculated with Escherichia coli. Journal-of-Food-Science. 2003; 68(3): 1003-1007

White, G. C. 1972. Hand book of chlorination. p. 596. Van Nostrand Reinhold Co., New York.

What is claimed is:

1. A method for sterilizing an aseptic processing line comprising the steps of generating chlorine dioxide gas, introducing the chlorine dioxide gas inside the aseptic processing line as a sterilization step, and removing the chlorine dioxide gas from the aseptic processing line.

2. The method of claim 1 wherein the aseptic processing line comprises at least one processing apparatus connected to a piping network.

3. The method of claim 2 wherein the processing apparatus can be a filter, heater, cooler, product filler, surge tank, and/or packager.

4. The method of claim 1 wherein the generating of the chlorine dioxide gas uses a gas generator.

5. The method of claim 1 wherein the generating of the chlorine dioxide gas uses a chlorine dioxide solution vaporized with a carrier gas.

6. The method of claim 5 wherein the carrier gas is air or nitrogen.

7. The method of claim 1 comprising connecting the aseptic line in a closed chlorine dioxide treatment loop to carry out the sterilization step.

8. The method of claim 7 further comprising flowing the chlorine dioxide gas in the loop to reduce microorganisms.

9. The method of claim 1 wherein the sterilization step uses bleeders to flow the gas.

10. The method of claim 1 wherein the sterilization step has a time period of about 30 minutes to about 1 hour.

11. The method of claim 1 wherein the atmosphere in the aseptic processing line has during the sterilization step a concentration of chlorine dioxide between about 1 to about 30 mg/L, a relative humidity between about 80 to about 95%, and a temperature between about 4 to about 45° C.

12. The method of claim 1 wherein the atmosphere in the aseptic processing line has during the sterilization step a concentration of chlorine dioxide between about 3 to about 10 mg/L, a relative humidity above 85%, and a temperature between about 3 to about 25° C.

13. The method of claim 11 further including flowing steam into the line to adjust the relative humidity in the line.

14. The method of claim 1 further comprising circulating the chlorine dioxide gas in the line during the sterilization step.

15. The method of claim 1 wherein the removing of the chlorine dioxide gas comprises flowing a secondary gas into the line.

16. The method of claim 15 wherein the secondary gas is nitrogen, air, or a mixture thereof.

17. The method of claim 1 wherein the removing step comprises using a blower to blow the chlorine dioxide gas from the line.

18. The method of claim 1 wherein the removing step comprises flowing water through the line.

19. The method of claim 1 wherein the water is heated.

20. The method of claim 1 further comprising monitoring the chlorine dioxide concentration in the line.

21. The method of claim 1 further comprising recycling the chlorine dioxide using a recycling device.

22. The method of claim 1 further comprising neutralizing the chlorine dioxide gas using a neutralizing device.

23. The method of claim 22 wherein the neutralizing device flows the chlorine dioxide gas through a reducing agent.

24. The method of claim 23 wherein the reducing agent is a soda lime, sodium thiosulfate, sodium sulfite, or a mixture thereof.

25. The method of claim 22 wherein the neutralizing device flows the chlorine dioxide gas through a gas scrubber.

26. The method of claim 1 further comprising flowing sterile air through the line after the sterilization and removal steps to maintain the sterilized surfaces in the line.

27. System for sterilizing an aseptic processing line comprising, a generator for generating chlorine dioxide gas, a device for introducing the chlorine dioxide gas inside the aseptic processing line, and a device for removing the chlorine dioxide gas from the aseptic processing line.

28. The system of claim 27 wherein the aseptic processing line comprises at least one processing apparatus connected to a piping network.

29. The system of claim 28 wherein the processing apparatus can be a filter, heater, cooler, product filler, surge tank, and/or packager.

30. The system of claim 27 wherein a gas generator generates the chlorine dioxide gas is from a generator.

31. The system of claim 27 wherein a generator is provided for generating the chlorine dioxide gas from a chlorine dioxide solution vaporized with a carrier gas.

32. The system of claim 27 that includes a chlorine dioxide gas treatment loop in which the aseptic line is connected to carry out the sterilization thereof.
